(19) United States
(12) Patent Application Publication
(10) Pub. No.: US 2026/0111682 A1
(43) Pub. Date: Apr. 23, 2026

(54) ARTIFICIAL INTELLIGENCE AVATAR INTERFACE FOR RESPONDING TO MEDICAL QUERIES USING REGULATORY-APPROVED CURATED CONTENT

(71) Applicant: ValueDo Life Sciences Advisory LLC, Albany, NY (US)

(72) Inventor: Gaurav Kandhari, New York, NY (US)

(21) Appl. No.: 19/284,743

(22) Filed: Jul. 30, 2025

Related U.S. Application Data
(60) Provisional application No. 63/709,030, filed on Oct. 18, 2024.

(51) Int. Cl. G06F 40/35; G16H 80/00

(52) U.S. Cl. CPC G06F 40/35; G16H 80/00

(57) ABSTRACT

Disclosed is a method and a system of an artificial intelligence avatar interface for responding to medical queries using regulatory-approved curated content. According to one embodiment, the method includes configuring a content delivery platform to receive approved content from a content creator and a regulatory body, coupling the content delivery platform to an LLM adapted to perform operations in a vector embedding space on a tokenized linguistic units within the approved content, configuring an avatar generation service to generate an AI avatar determined as a function of the curated topic set, and receiving a voice input and a text input comprising a user question directed to the generated AI avatar. In response to determining the user question is not answerable, marking the user question as unanswerable in a database and sending an indication to the content creator and the regulatory body that the user question is unanswerable.

### CLAIM OF PRIORITY

This Application is a Conversion Application of, claims priority to, and incorporates by reference herein the entirety of the disclosure of U.S. Provisional Ser. No. 63/709,030 titled JAWAAB-FIRST AI EMPOWERED HCP PERSONAL CONCIERGE filed on Oct. 18, 2024.

### FIELD OF TECHNOLOGY

This disclosure relates generally to data processing devices and, more particularly, to a method, a device and/or a system of an artificial intelligence avatar interface for responding to medical queries using regulatory-approved curated content.

### BACKGROUND

Pharmaceutical companies may face significant challenges in delivering information to Healthcare Providers (HCPs), including but not limited to relying heavily on sending sales representatives, printed brochures, and/or static websites, which often prove inefficient. These approaches may be time-consuming, expensive, and/or limited in reach, which may hinder the timely dissemination of crucial information about new drugs and/or updates. This may result in delays in information delivery, reduced engagement with HCPs, and/or potential misinterpretation of important details.

The static and/or fragmented nature of current content delivery platforms may further limit the ability of pharmaceutical companies to effectively monitor and/or understand how users are interacting with the content delivery platforms. Companies may be unable to determine which questions are frequently asked, whether the right information is being accessed, or how effectively the content delivery platforms address the needs of healthcare providers and/or patients. In some instances, important questions may go unanswered due to the lack of a dynamic and/or responsive system.

Moreover, the process of updating and maintaining approved content may be time-consuming and/or prone to error when managed manually across multiple platforms. These difficulties may be compounded by strict compliance requirements, which may limit the use of certain technologies and/or impose restrictions on the way information is displayed, shared, and/or customized.

In addition, many digital tools may not give companies enough insight into what Healthcare Providers are asking and/or how well the system is working. Without this feedback, it may be difficult to improve the AI experience and/or make sure it is actually helping users. Complex login systems may also create barriers for busy HCPs, who may not want to remember multiple passwords and/or go through difficult authentication steps just to get basic information. This may reduce overall engagement and limit the tool's effectiveness.

### SUMMARY

Disclosed are a method, a device and/or a system of an artificial intelligence avatar interface for responding to medical queries using regulatory-approved curated content.

In one aspect, a method includes configuring a content delivery platform to receive approved content. The approved content has been approved by a content creator and/or a regulatory body. The method includes coupling the content delivery platform to an LLM adapted to perform operations in a vector embedding space on a tokenized linguistic unit within the approved content. The approved content includes a question, an answer, an image, a video, an audio, a document, a web page, a link, and/or a markup, using the content delivery platform. The method includes characterizing one or more topics in the approved content using the LLM to extract the topics from the approved content. The method includes creating a curated topic set based on identifying and/or eliminating mutually exclusive topics from the extracted topics using the LLM. The method includes creating a curated question and answer set including mutually exclusive questions and/or answers from the curated topic set. The mutually exclusive questions and/or answers are determined as a function of the curated topic set using the LLM. The method includes configuring an avatar generation service to generate an AI avatar determined as a function of the curated topic set, using the content delivery platform, and providing a user with interactive access to the generated avatar, using the content delivery platform. The method includes receiving a voice input and/or a text input, and includes a user question directed to the generated AI avatar, using the content delivery platform. The method includes determining if the user question is answerable by an answer selected from the curated question and answer set based on comparing a threshold distance to a distance between the user question and/or each question from the curated question and answer set in the vector embedding space, using the LLM. The method includes in response to determining the user question is not answerable, marking the user question as unanswerable in a database and/or sending an indication to the content creator and/or the regulatory body that the user question could not be answered, and/or in response to determining the user question is answerable finding a best answer to the user question. The best answer is selected from the curated question and answer set as a function of a distance in the vector embedding space using the LLM and/or directing the generated avatar to interactively answer the user question with the selected best answer to the user question using the content delivery platform. The method may further include, in response to determining that two or more questions are present in the received voice and/or text input, using the LLM to find the best answer to the two or more questions. The best answer may be selected from a curated question-and-answer set as a function of the distance between answers in the vector embedding space, using the LLM. The method may then direct the generated avatar to interactively respond to the user with the selected best answer. The generated avatar may present content that is most relevant to the user question, where the relevant content is determined based on the distance between a speculative content item and/or the selected best answer in the vector embedding space, using the LLM.

The method may include presenting the content that is most relevant. The relevant content may further include a slide presentation. The slide presentation may include a sequence of slides ordered as a function of the minimum distance from slide to slide in the vector embedding space using the LLM. The method may further include providing a downloadable document, where the document is selected as a function of the minimum distance from the selected best answer in the vector embedding space using the LLM. The user may be presented with the option to download the content to their device for future reference. The method may further include presenting a follow-up question to the user. The follow-up question may be directed to determining whether the user would like more information about a subtopic closely related to the selected best answer. The subtopic may be determined as a function of the selected best answer and/or a curated question-and-answer set using the LLM.

A system includes a content delivery platform that comprises a user interface configured to receive a user question and to deliver a best answer. The content delivery platform includes a tablet, a desktop, and/or a mobile device, each configured to receive and/or transmit the user question to an artificial intelligence (AI) engine. A large language model is integrated within the AI engine and is communicatively coupled to the content delivery platform. The large language model is configured to perform operations in a vector embedding space on a tokenized linguistic unit derived from approved content. The AI engine is configured to process the user question received via a voice input and/or a text input from the content delivery platform. It retrieves relevant information from a database that includes the approved content. The approved content includes a curated topic set derived from extracted topics using the large language model, and/or a curated question and answer set that includes mutually exclusive questions and/or mutually exclusive answers derived from the curated topic set. The approved content is presented in one or more formats selected from an image, a video, an audio file, a document, a web page, a hyperlink, and/or a markup. The system compares a threshold distance to an actual distance between the user question and/or each question from the curated question and answer set in the vector embedding space. It identifies the best answer based on a minimum distance below the threshold distance. If the actual distance is determined to be within the threshold distance, the system generates the best answer to the user question and/or transmits the best answer to the content delivery platform.

An avatar generation service is communicatively coupled to the AI engine and/or the content delivery platform. The avatar generation service is configured to generate and/or manage an AI avatar for interaction with the user through the content delivery platform. It may also direct the AI avatar to interactively answer the user question using the selected best answer from the curated question and answer set. The best answer is selected using the vector embedding space by the AI engine. A feedback loop is configured to transmit data from user interactions with the artificial intelligence engine via the content delivery platform. The transmitted data is processed to continuously refine the curated question and answer set and to enhance the response generation capabilities of the large language model. Each user question is tagged with a feedback label indicating whether the question was answered, unanswered, requires revision, or any combination thereof. The tagged question is stored in the database for continuous training of the large language model. The curated question and answer set is updated based on feedback received from users, content creators, and/or a regulatory body. The feedback is processed by the large language model to refine the curated question and answer set over time.

The system may receive the user question through a voice input and/or a text input. The artificial intelligence engine may be configured to convert the voice input and/or the text input into a standardized format before processing the user question within a vector embedding space. The AI avatar may be further configured to present to the user content that is most relevant in the context of the user question. The relevant content may include a slide presentation and/or a downloadable document 212, selected as a function of a minimum distance from the selected best answer in the vector embedding space. The feedback loop may be further configured to process the tagged user questions to identify recurring patterns in unanswered questions. The large language model may adjust the curated question and answer set to improve coverage and/or enhance the relevance of future responses. The system may further include a peer-to-peer (P2P) sharing module integrated into the content delivery platform. The P2P sharing module may be configured to allow the user to share approved content by selecting a share option from a dropdown menu. The module may generate a QR code and/or a tokenized invite link associated with the approved content. The user may be enabled to share either the QR code, the tokenized invite link, and/or both, to provide a peer with access to the content delivery platform through a two-click interaction.

A method includes identifying a natural language input in the form of a user question transmitted by a user. The method further includes providing the natural language input of the user question to a large language model. The large language model operates within a vector embedding space that processes a tokenized linguistic unit derived from medical and/or pharmacological phraseology. The method includes analyzing the natural language text of the user question using the large language model. The large language model compares a first threshold distance to an actual distance between the user question and each question from a curated question and answer set in the vector embedding space. The method includes determining whether the actual distance is within the first threshold distance. If the actual distance is determined to be within the first threshold distance, the method further includes automatically generating a best answer as a responsive communication to the user as an output of the large language model. The method includes transmitting the responsive communication to the user.

A method may involve analyzing a curated topic set using a large language model to extract relevant topics or relationships. Subsequently, a curated question and answer set is created as an output of the large language model's analysis of this topic set, with the question and/or answer set generated based on the identified topics or relationships. The method may also include tagging a user's question as unanswerable if its actual distance falls outside a first threshold distance. This unanswerable question can then be marked in a database, and/or a content creator may be notified. This notification allows the content creator to involve a regulatory body if new content requires approval to answer the previously unanswerable question. Furthermore, the method may involve transmitting a responsive communication to the user via an AI avatar that interacts with them on a content delivery platform. This AI avatar is generated based on the curated question and answer set, and is configured to interactively present the selected best answer to the user through the content delivery platform. Finally, the method may encompass receiving user questions through various formats, including voice input, text input, or a selection from displayed questions on the user interface of the content delivery platform. The large language model is adapted to process each format, converting voice input into text input and then analyzing the text input to determine the best answer by comparing the actual distance and a threshold distance within a vector embedding space.

A method may involve selecting the best answer to a user question from a curated question and answer set based on both semantic relevance and contextual appropriateness. The semantic relevance is determined by comparing the user questions vector representation with the vectors of questions in the curated set within a vector embedding space. Conversely, the contextual appropriateness is determined by considering the context of the user's specific data. The method may also include updating the curated question and answer set based on feedback received from users and/or content creators. This feedback is processed by a large language model to refine and improve the curated question and answer set over time, thereby increasing the accuracy and relevance of future responses.

A method may involve tagging each user question with a feedback label indicating whether the question was answered, unanswered, or requires revision. These tagged user questions can then be stored in a database for continuous training of the large language model. The large language model utilizes these tagged questions to track the performance of the AI engine and refine its question-answering capabilities. By processing the tagged user questions, the system can identify recurring patterns in unanswered questions. Subsequently, the large language model adjusts the curated question and answer set to address identified gaps, improve coverage, or enhance the relevance of future responses.

The method may also include detecting and processing multiple user questions within a single input. The large language model analyzes the natural language input to segment it into individual questions and then generates a responsive communication to the user that answers each segmented question using the curated question and answer set, based on the distances between the user questions and answers in the vector embedding space.

Furthermore, the method may involve detecting trigger words indicative of an adverse event in the user's input using the large language model. It can then generate a dialog box through the AI avatar to confirm whether the user experienced the event and/or reported the adverse event. The system may then receive input from the user in the form of a “YES” or “NO” response, store the user's response in the database, and/or transmit an alert to a patient safety team to initiate an action.

The method may also include identifying trigger phrases in the user's input suggestive of a product quality issue related to a pharmaceutical product using the large language model. It can then present a dialogue box to the user via the AI avatar to verify if the product quality issue was experienced. The system may collect the user's response, store it in the database, and/or inform the patient safety team to take appropriate further action.

Finally, the method may involve alerting the user that their question is unanswerable if the actual distance is outside of the first threshold distance. This unanswerable alert can be conveyed via the AI avatar. The system may also present the user with a medical science liaison option if the user question is unanswerable using the AI avatar. If the user accepts this option, it prompts the AI engine to alert the medical science liaison that the user has at least one unanswerable question and would like to be contacted.

The methods and systems disclosed herein may be implemented in any means for achieving various aspects, and may be executed in a form of a non-transitory machine-readable medium embodying a set of instructions that, when executed by a machine, cause the machine to perform any of the operations disclosed herein. Other features will be apparent from the accompanying drawings and from the detailed description that follows.

### BRIEF DESCRIPTION OF THE DRAWINGS

The embodiments of this invention are illustrated by way of example and not limitation in the figures of the accompanying drawings, in which like references indicate similar elements and in which:

FIG. 1 illustrates the network view of an AI-powered concierge system which encompasses the overall system and how its various components interact, according to one embodiment.

FIG. 2 is a system architecture view of the AI-powered concierge system designed to provide information to a user, according to one embodiment.

FIG. 3 is a user interaction and question answering workflow illustrating a step-by-step process flow that a user follows to interact with the AI-powered concierge system of FIG. 1, and receive answers to questions, according to one embodiment.

FIG. 4 is a schematic diagram of AI-driven information retrieval of FIG. 1, according to one embodiment.

FIG. 5 is an avatar-mediated communication to illustrate how the AI avatar facilitates communication and delivers information to the user within the AI-powered concierge system FIG. 1, according to one embodiment.

FIG. 6 illustrates a content improvement via a feedback loop showcasing how the AI-powered concierge system FIG. 1, manages user feedback to improve the content and responses over time, according to one or more embodiments.

FIG. 7 illustrates a story-driven example to explain how a user interacts with and benefits from the AI-powered concierge system of FIG. 1-6, according to one embodiment.

Other features of the present embodiments will be apparent from the accompanying drawings and from the detailed description that follows.

### DETAILED DESCRIPTION

Example embodiments, as described below, may be used to provide a method, a system and/or a device of an artificial intelligence avatar interface for responding to medical queries using regulatory-approved curated content.

In one embodiment, a method includes configuring a content delivery platform 102 to receive approved content 114. The approved content 114 has been approved by a content creator 116 and/or a regulatory body 118. The method includes coupling the content delivery platform 102 to a large language model LLM 146 adapted to perform operations in a vector embedding space 136 on a tokenized linguistic unit 138 within the approved content 114. The approved content 114 includes a question and answer set 130, an image, a video, an audio, a document, a web page, a link, and/or a markup, using the content delivery platform 102. The method includes characterizing one or more topics in the approved content 114 using the LLM 146 to extract a curated topic set 128. The method includes creating the curated topic set 128 based on identifying and/or eliminating mutually exclusive topics using the LLM 146. The method includes creating a curated question and answer set 130, comprising mutually exclusive questions and/or answers from the curated topic set 128. The mutually exclusive questions and/or answers are determined as a function of the curated topic set 128 using the LLM 146.

The method includes configuring an avatar generation service 202 to generate an AI avatar 204 determined as a function of the curated topic set 128, and providing a user 112 with interactive access to the AI avatar 204 via the content delivery platform 102. The method includes receiving a voice/text input 206, and includes a user question 124 directed to the AI avatar 204, using the content delivery platform 102. The method includes determining if the user question 124 is answerable by an answer selected from the curated question and answer set 130, based on comparing a threshold distance 134 to an actual distance 402 between the user question 124 and each question in the curated question and answer set 130 in the vector embedding space 136, using the LLM 146. The method includes in response to determining the user question 124 is not answerable, marking the user question 124 as unanswerable in a database 126 and/or sending an indication to the content creator 116 and/or the regulatory body 118 that the user question 124 could not be answered, and/or in response to determining the user question 124 is answerable finding a best answer 144 to the user question 124. The best answer 144 is selected from the curated question and answer set 130 as a function of a distance in the vector embedding space 136, using the LLM 146 and/or directing the generated avatar to interactively answer the user question 124 with the selected best answer 144 to the user question 124 using the content delivery platform 102.

The method may further include, in response to determining that two or more questions are present in the received voice and/or text input 206, using the LLM 146 to find the best answer 144 to the two or more questions. The best answer 144 may be selected from a curated question and answer set 130 as a function of the distance between answers in the vector embedding space 136, using the LLM 146. The method may then direct the generated avatar to interactively respond to the user 112 with the selected best answer 144. The generated avatar may present content that is most relevant to the user question 124, where the relevant content is determined based on the distance between a speculative content item and/or the selected best answer 144 in the vector embedding space 136, using the LLM 146.

The method may include presenting the content that is most relevant. The relevant content may further include a slide presentation 210. The slide presentation 210 may include a sequence of slides ordered as a function of the minimum distance 140 from slide to slide in the vector embedding space 136 using the LLM 146. The method may further include providing a downloadable document 212, where the document is selected as a function of the minimum distance 140 from the selected best answer 144 in the vector embedding space 136 using the LLM 146. The user 112 may be presented with the option to download the content to their device for future reference. The method may further include presenting a follow-up question to the user 112. The follow-up question may be directed to determining whether the user 112 would like more information about a subtopic closely related to the selected best answer 144. The subtopic may be determined as a function of the selected best answer 144 and/or a curated question and answer set 130 using the LLM 146.

A system includes a content delivery platform 102 that comprises a user interface 208 configured to receive a user question 124 and to deliver a best answer 144. The content delivery platform 102 includes a tablet 104, a desktop 106, and/or a mobile device 108, each configured to receive and/or transmit the user question 124 to an artificial intelligence (AI) engine 120. A large language model 146 is integrated within the AI engine 120 and is communicatively coupled to the content delivery platform 102. The large language model 146 is configured to perform operations in a vector embedding space 136 on a tokenized linguistic unit 138 derived from approved content. The AI engine 120 is configured to process the user question 124 received via a voice input and/or a text input 206 from the content delivery platform 102. It retrieves relevant information from a database 126 that includes the approved content 114.

The approved content 114 includes a curated topic set 128 derived from extracted topics using the large language model 146, and/or a curated question and answer set 130 that includes mutually exclusive questions and/or mutually exclusive answers derived from the curated topic set 128. The approved content 114 is presented in one or more formats selected from an image, a video, an audio file, a document, a web page, a hyperlink, and/or a markup. The system compares a threshold distance 134 to an actual distance 402 between the user question 124 and/or each question from the curated question and answer set 130 in the vector embedding space 136. It identifies the best answer 144 based on a minimum distance 140 below the threshold distance 134. If the actual distance 402 is determined to be within the threshold distance 134, the system generates the best answer 144 to the user question 124 and/or transmits the best answer 144 to the content delivery platform 102.

An avatar generation service 202 is communicatively coupled to the AI engine 120 and/or the content delivery platform 102. The avatar generation service 202 is configured to generate and/or manage an AI avatar 204 for interaction with the user 112 through the content delivery platform 102. It may also direct the AI avatar 204 to interactively answer the user question 124 using the selected best answer 144 from the curated question and answer set. The best answer 144 is selected using the vector embedding space 136 by the AI engine 120. A feedback loop 148 is configured to transmit data from user interactions with the artificial intelligence engine 120 via the content delivery platform 102. The transmitted data is processed to continuously refine the curated question and answer set 130 and to enhance the response generation capabilities of the large language model 146. Each user question 124 is tagged with a feedback label indicating whether the question was answered, unanswered, requires revision, or any combination thereof. The tagged question is stored in the database 126 for continuous training of the large language model 146. The curated question and answer set 130 is updated based on feedback received from a user 112, a content creators 116, and/or a regulatory body 118. The feedback is processed by the large language model 146 to refine the curated question and answer set 130 over time.

The system may receive the user question 124 through a voice input and/or a text input 206. The artificial intelligence engine 120 may be configured to convert the voice input and/or the text input 206 into a standardized format before processing the user question 124 within a vector embedding space 136. The AI avatar 204 may be further configured to present to the user content that is most relevant in the context of the user question 124. The relevant content may include a slide presentation 210 and/or a downloadable document 212, selected as a function of a minimum distance 140 from the selected best answer 144 in the vector embedding space 136. The feedback loop 148 may be further configured to process the tagged user questions 124 to identify recurring patterns in unanswered questions. The large language model 146 may adjust the curated question and answer set 130 to improve coverage and/or enhance the relevance of future responses. The system may further include a peer-to-peer (P2P) sharing module 214 integrated into the content delivery platform 102. The P2P sharing module 214 may be configured to allow the user 112 to share approved content 114 by selecting a share option from a dropdown menu 216. The module may generate a QR code 218 and/or a tokenized invite link 220 associated with the approved content 114. The user 102 may be enabled to share either the QR code 218, the tokenized invite link 220, and/or both, to provide a peer with access to the content delivery platform 102 through a two-click interaction.

A method includes identifying a natural language input in the form of a user question 124 transmitted by a user 102. The method further includes providing the natural language input of the user question 124 to a large language model 146. The large language model 146 operates within a vector embedding space 136 that processes a tokenized linguistic unit 138 derived from medical and/or pharmacological phraseology. The method includes analyzing the natural language text of the user question 124 using the large language model 146. The large language model 146 compares a first threshold distance 134 to an actual distance 402 between the user question 124 and each question from a curated question and answer set 130 in the vector embedding space 136. The method includes determining whether the actual distance 402 is within the first threshold distance 134. If the actual distance 402 is determined to be within the first threshold distance 134, the method further includes automatically generating a best answer 144 as a responsive communication 142 to the user 112 as an output of the large language model 146. The method includes transmitting the responsive communication 142 to the user 102.

A method may involve analyzing a curated topic set 128 using a large language model 146 to extract relevant topics or relationships. Subsequently, a curated question and answer set 130 is created as an output of the large language model's 146 analysis of this topic set, with the question and answer set 130 generated based on the identified topics or relationships. The method may also include tagging a user question 124 as unanswerable if its actual distance 402 falls outside a first threshold distance 134. This unanswerable question can then be marked in a database 126, and a content creator 116 may be notified. This notification allows the content creator 116 to involve a regulatory body 118 if new content requires approval to answer the previously unanswerable question. Furthermore, the method may involve transmitting a responsive communication 142 to the user 102 via an AI avatar 204 that interacts with them on a content delivery platform 102. This AI avatar 204 is generated based on the curated question and answer set 130, and is configured to interactively present the selected best answer 144 to the user 102 through the content delivery platform 102. Finally, the method may encompass receiving user questions 124 through various formats, including voice input, text input, or a selection from displayed questions on the user interface 208 of the content delivery platform 102. The large language model is adapted to process each format, converting voice input into text input and then analyzing the text input to determine the best answer 144 by comparing the actual distance 402 and a threshold distance within a vector embedding space 136.

A method may involve selecting the best answer 144 to a user question 124 from a curated question and answer set 130 based on both semantic relevance and contextual appropriateness. The semantic relevance is determined by comparing the user questions 124 vector representation with the vectors of questions in the curated set within a vector embedding space 136. Conversely, the contextual appropriateness is determined by considering the context of the user's 102 specific data. The method may also include updating the curated question and answer set 130 based on feedback received from the users 102 and/or the content creators 116. This feedback is processed by a large language model 146 to refine and improve the curated question and answer set 130 over time, thereby increasing the accuracy and relevance of future responses.

A method may involve tagging each user question 124 with a feedback label indicating whether the question was answered, unanswered, or requires revision. These tagged user questions 124 can then be stored in a database 126 for continuous training of the large language model 146. The large language model 146 utilizes these tagged questions to track the performance of the AI engine 120 and refine its question-answering capabilities. By processing the tagged user questions 124, the system can identify recurring patterns in unanswered questions. Subsequently, the large language model 146 adjusts the curated question and answer set 130 to address identified gaps, improve coverage, or enhance the relevance of future responses.

The method may also include detecting and processing multiple user questions 124 within a single input. The large language model 146 analyzes the natural language input to segment it into individual questions and then generates a responsive communication 142 to the user 102 that answers each segmented question using the curated question and answer set 130, based on the distances between the user questions 124 and answers in the vector embedding space 136.

Furthermore, the method may involve detecting trigger words indicative of an adverse event in the user's input using the large language model 146. It can then generate a dialog box through the AI avatar 204 to confirm whether the user 102 experienced the event and/or reported the adverse event. The system may then receive input from the user 102 in the form of a “YES” or “NO” response, store the user's response in the database 126, and/or transmit an alert to a patient safety team to initiate an action.

The method may also include identifying trigger phrases in the user's input suggestive of a product quality issue related to a pharmaceutical product using the large language model 146. It can then present a dialogue box to the user 102 via the AI avatar 204 to verify if the product quality issue was experienced. The system may collect the user's response, store it in the database, and/or inform the patient safety team to take appropriate further action.

Finally, the method may involve alerting the user 102 that their question is unanswerable if the actual distance 402 is outside of the first threshold distance. This unanswerable alert can be conveyed via the AI avatar 204. The system may also present the user 102 with a medical science liaison option if the user question 124 is unanswerable using the AI avatar 204. If the user 102 accepts this option, it prompts the AI engine 120 to alert the medical science liaison that the user 102 has at least one unanswerable question and would like to be contacted.

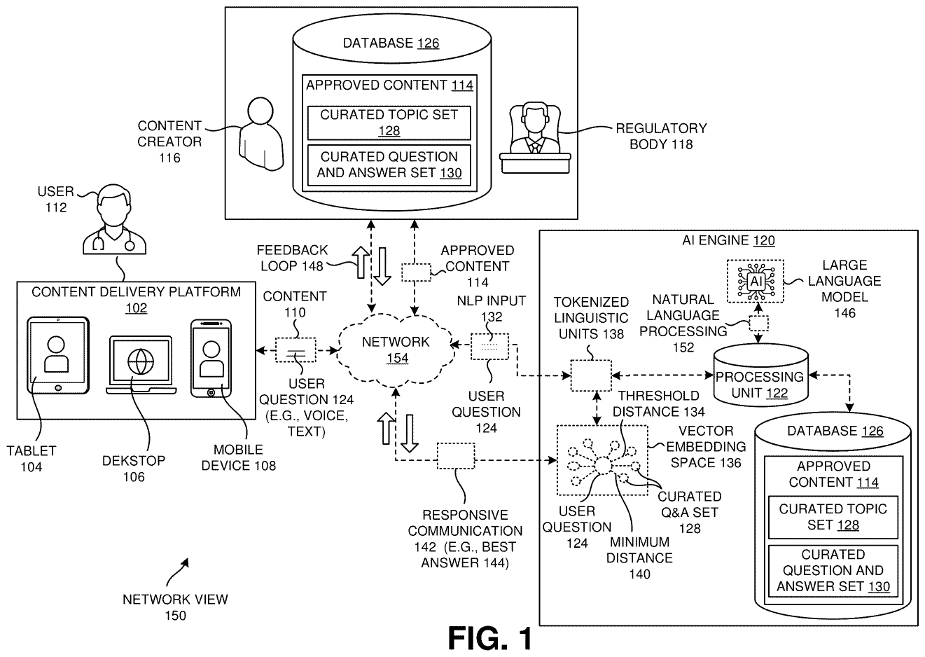

FIG. 1 illustrates the network view 150 of an AI-powered concierge system comprising a content delivery platform 102, a tablet 104, a desktop 106, a mobile device 108, a content 110, a user 112, an approved content 114, a content creator 116, a regulatory body 118, an AI engine 120, a processing unit 122, an user question 124, a database 126, a curated topic set 128, a curated question and answer set 130, a NLP input 132, a threshold distance 134, a vector embedding space 136, a tokenized linguistic units 138, a minimum distance 140, a responsive communication 142, a best answer 144, a large language model 146, a feedback loop 148, and a natural language processing 152, according to one embodiment.

The content delivery platform 102 may be a centralized system configured to receive the approved content 114 that may be validated by at least one of a content creator 116 and a regulatory body 118. The content delivery platform 102 may include the tablet 104, a desktop 106, and/or the mobile device 108, each adapted to receive the user question 124 via the voice/text input 206. The content delivery platform 102 may transmit the user question 124 to the large language model 146 integrated within the AI engine 120, which may operate in the vector embedding space 146 on the tokenized linguistic units 138 derived from the approved content 114. The content delivery platform 102 may support the extraction of topics using the large language model 146 to generate the curated topic set 128 and the curated question and answer set 130, comprising mutually exclusive questions and answers. The content delivery platform 102 may further control the avatar generation service 202 to generate the AI avatar 204 based on the curated topic set 128 and provide interactive responses to the user questions 124. Based on a distance comparison in the vector embedding space 136, the content delivery platform 102 may direct the AI avatar 204 to answer the user question 124 and/or trigger an alert to the content creator 116 and/or the regulatory body 118 if the question is unanswerable, according to one embodiment.

The tablet 104 may be a touch-enabled access point that may allow the user 112 to interact with the content delivery platform 102. The tablet 104 may transmit the user question 124 to the content delivery platform 102 for processing by the AI engine 120. The tablet 104 may display the responsive communication 142, including the best answer 144, in a user-friendly format. The tablet 104 may connect via the network 154 and may support both text and/or voice-based inputs for submission to the AI engine 120, according to one embodiment.

The desktop 106 may be a browser-based user interface 208 that may allow the user 112 to access the content delivery platform 102 using a desktop and/or laptop computer. The desktop 106 may support natural language inputs, including but not limited to text queries, document uploads, and/or hyperlinks, and may forward these inputs to the AI engine 120 for the natural language processing 152. The desktop 106 may retrieve the responsive communication 142 from the approved content 114 and may display curated content, including but not limited to pharmaceutical products and/or medical guidelines. The desktop 106 may also serve as a hub for collecting user feedback for the system's learning loop, according to one embodiment.

The mobile device 108 may be a smartphone-optimized application that may allow the user 112 to submit the user question 124 directly to the content delivery platform 102. The mobile device 108 may facilitate quick access to the responsive communication 142 and may be designed for on-the-go decision support by clinicians and/or healthcare workers. The mobile device 108 may operate with the AI engine 120 through the same underlying network and may include voice-to-text features, alerts, and/or simplified access to the approved content 114, according to one embodiment.

The content 110 may be any tangible and/or digital information, including but not limited to text, images, audio, video, documents, web pages, links, or structured data, that may be created, modified, stored, transmitted, and/or displayed by one or more systems for purposes including but not limited to, communication, reference, analysis, and/or interaction. The content 110 may be generated by content creators 116 and/or the regulatory body 118. The content 110 may serve as the raw material from which the curated topic set 128 and the approved content 114 may be derived. The content 110 may not necessarily be validated for delivery to the user 112 until it is reviewed and/or marked as the approved content 114. The content 110 may be stored in the database 126 and accessed by the AI engine 120 to process, evaluate, and/or compare with the user questions 124 in the vector embedding space 136, according to one embodiment.

The user 112 may provide the user question 124 via a text input, voice input, or by selecting a predefined query through the user interface 208. The user 112 may interact with the AI avatar 204 generated by the avatar generation service 202 and may receive the responsive communication 142, including the best answers 148 and/or relevant content 110. The user 112 may also provide feedback that is processed through the feedback loop 148 to enhance the curated question and answer set 130, according to one embodiment.

The approved content 114 may be a validated and/or regulatory-compliant subset of the content 110. The approved content 114 may include one or more of a question, an answer, an image, a video, an audio file, a document, a web page, a hyperlink, and/or a markup. The approved content 114 may be reviewed and/or approved by at least one of the content creators 116 and the regulatory body 118 before being delivered to the user 112. The approved content 114 may be used by the large language model 146 to extract curated topics and generate the curated question and answer sets 130. The approved content 114 may be the only content eligible for matching in the vector embedding space 136 during the determination of the best answer 144 to the user question 124. The approved content 114 may be presented in response to a successful distance match and/or selected for download, avatar presentation, and/or further contextual interaction, according to one embodiment.

The content creator 116 may be an authorized entity and/or expert responsible for generating, reviewing, and curating medical and pharmaceutical content. The content creator 116 may approve the content 110 and/or contribute to the creation of the curated topic sets 128 and the curated question and answer set 130. The content creator 116 may also receive notifications when the user questions 124 are marked as unanswerable, allowing them to generate new and/or revised content for future approval. The content creator 116 may collaborate with the regulatory body 118 to ensure that the approved content 114 meets quality and/or compliance standards, according to one embodiment.

The regulatory body 118 may be an oversight authority responsible for reviewing and approving the content 110 developed by the content creator 116. The regulatory body 118 may ensure that all approved content 114 complies with legal, clinical, and/or industry-specific standards. The regulatory body 118 may also be notified when the user question 124 cannot be answered within the approved threshold, triggering content refinement and/or new content generation, according to one embodiment.

The regulatory body 118 may be an oversight entity responsible for ensuring the accuracy, legality, and clinical reliability of the approved content 114. The regulatory body 118 may review and/or approve curated content generated by the content creators 116. The regulatory body 118 may receive notifications if the user question 124 cannot be answered, particularly in contexts that may require new clinical validation and/or patient safety review. The regulatory body 118 may serve as a final gatekeeper for the content 110 before it becomes available as the approved content 114 within the content delivery platform 102, according to one embodiment.

The AI engine 120 may be a computational component responsible for processing natural language inputs and/or performing semantic operations. The AI engine 120 may receive the user question 124 via the content delivery platform 102 and may tokenize the input, convert it to a vector format, and compare it to a curated question and answer set 130 using the large language model 146. The AI engine 120 may also direct the responsive communication 142 and interact with the feedback loop 148 to improve future answers, according to one embodiment.

The processing unit 122 may be the hardware and/or logical module that executes AI operations. The processing unit 122 may perform natural language processing 152 transformations, calculate distances within the vector embedding space 136, and determine whether the user question 124 falls within the threshold distance 134 for selecting the best answer 144. The processing unit 122 may serve as the execution core of the AI engine 120, according to one embodiment.

The user question 124 may be a natural language inquiry received from the user 112. The user question 124 may be submitted via the voice/text input 206 using the content delivery platform 102. The content delivery platform 102 may transmit the user question 124 to the AI engine 120, which may process the input using natural language processing 152 to generate the tokenized linguistic units 138. The AI engine 120, utilizing the large language model 146, may compare the vector representation of the user question 124 to questions stored in the curated question and answer set 130 within the database 126. If the user question 124 does not fall within the threshold distance 134 from any stored question, the AI engine 120 may tag the user question 124 as unanswerable and store it in the database 126 for further review by the content creator 116 and/or the regulatory body 118, according to one embodiment.

The database 126 may be a structured storage environment that holds system content/information. The database 126 may store, including but not limited to the approved content 114, the curated topic sets 134, the curated question and answer sets 130, and/or the tagged user questions 124. The database 126 may also retain feedback labels for continuous training of the large language model 146, according to one embodiment.

The curated topic set 128 may be a collection of medical topics identified and/or refined from the approved content 114. The curated topic set 128 may be generated by the large language model 146 by analyzing extracted topics and/or eliminating mutual redundancies. It may serve as the foundation for creating mutually exclusive questions and answers in the curated question and answer set 130, according to one embodiment.

The curated question and answer set 130 may be a collection of validated question-answer pairs. The curated question and answer set 130 may be derived from the curated topic set 128 and stored in the database 126. The curated question and answer set 130 may be used by the AI engine 120 to match the user questions 124 based on semantic distance and select the best answer 144, according to one embodiment.

The NLP input 132 may be the normalized language representation of the user's 112 query. The NLP input 132 may be produced by parsing and/or tokenizing the user question 124, which may then be converted into the embedding vector for comparison within the vector embedding space 136, according to one embodiment.

The threshold distance 134 may be a predefined similarity value that defines match relevance. The AI engine 120 may use the threshold distance 134 to determine if the vectorized user question 124 is close enough to any question in the curated question and answer set 130. If not, the input may be marked unanswerable, according to one embodiment.

The vector embedding space 136 may be a high-dimensional mathematical space for comparing text inputs. The vector embedding space 136 may allow the AI engine 120 to compare the user questions 130 to curated questions based on semantic similarity, which may be measured by calculating actual distance 402 values, according to one embodiment.

The tokenized linguistic units 138 may be discrete components derived from a natural language string. The tokenized linguistic units 138 may be produced from the user question 124 and may be fed into the large language model 146 for generating embedding vectors that represent the semantic meaning of the input, according to one embodiment.

The minimum distance 140 may be the shortest measured vector distance between the user question 124 and one or more entries in the curated question and answer set 130. If the minimum distance 140 falls below the threshold distance 134, the AI engine 120 may identify and retrieve the best answer 144 as the responsive communication 142, according to one embodiment.

The responsive communication 142 may be the output returned to the user 112 based on their query. The AI engine 120 may use the selected best answer 144 from the curated question and answer set 130 to generate the responsive communication 142, which may be delivered via the AI avatar 204 on the content delivery platform 102, according to one embodiment.

The best answer 144 may be the most relevant response identified from the curated question and answer set 130. The best answer 144 may be selected as a function of the minimum distance 140 within the vector embedding space 136 and delivered to the user 112 as the output of the content delivery platform 102, according to one embodiment.

The large language model 146 may be a trained neural network capable of understanding and generating language representations. The large language model 146 may perform semantic comparison of the user questions 124 to the content 110 stored in the database 126, generate the curated topic sets 134, curate the curated question and answer sets 136, and/or refine the responsive communication 142 delivered through the content delivery platform 102 based on ongoing feedback received via the feedback loop 148, according to one embodiment.

The feedback loop 148 may be a mechanism to collect and apply system learning from user interactions. The feedback loop 148 may process, including but not limited to, user satisfaction, content performance, and unanswered queries to update the curated question and answer set 130 and/or retrain the large language model 146 over time, according to one embodiment.

The natural language processing 152 may be the method of converting raw human language into computational representations. The natural language processing 152 may transform the user question 124 into structured input, including the NLP input 132 and/or tokenized linguistic units 138, which may then be processed by the AI engine 120 and/or the large language model 146 to calculate semantic similarities within the vector embedding space 136 and/or guide the retrieval of the best answer 144 from the curated question and answer set 130, according to one embodiment.

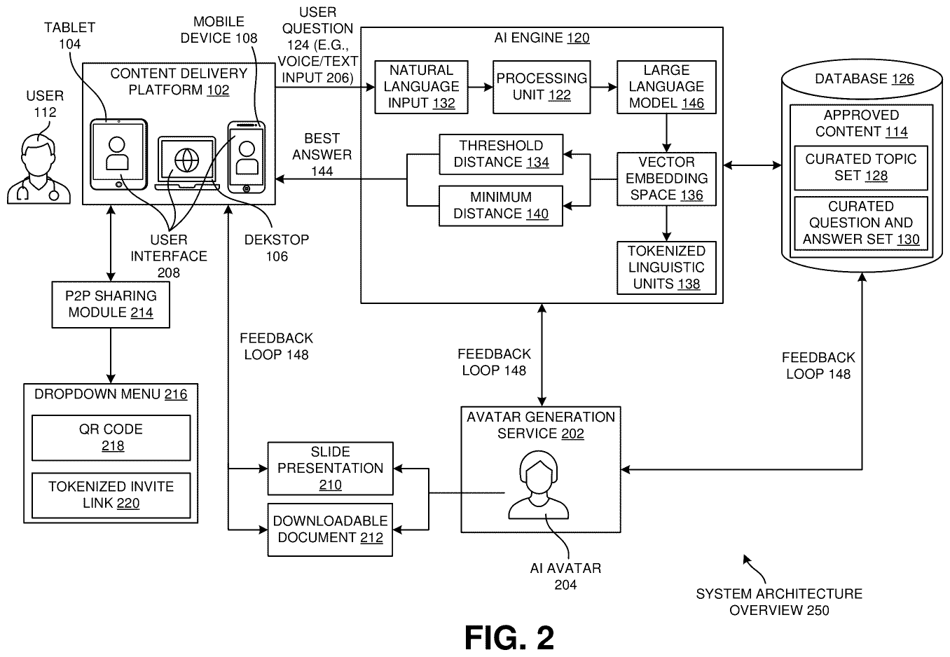

FIG. 2 is a system architecture view 250 of the AI-powered concierge system of FIG. 1, according to one embodiment. FIG. 2 illustrates the content delivery platform 102, the tablet 104, the desktop 106, the mobile device 108, the user 112, the approved content 114, the AI engine 120, the processing unit 122, the user question 124, the database 126, the curated topic set 128, the curated question and answer set 130, the NLP input 132, the threshold distance 134, the vector embedding space 136, the tokenized linguistic units 138, the minimum distance 140, the responsive communication 142, the best answer 144, the large language model 146, the feedback loop 148, an avatar generation service 202, an AI avatar 204, a voice/text input 206, a user interface 208, a slide presentation 210, a downloadable document 212, a P2P sharing module 214, a dropdown menu 216, a QR code 218, and a tokenized invite link 220, according to one embodiment.

The avatar generation service 202 may be a backend software module configured to dynamically generate the AI avatars 204 in response to the user question 124 received from the user 112. The avatar generation service 202 may utilize inputs including, but not limited to, natural language text, voice input, visual branding parameters, and/or curated topic categories to generate the AI avatars 204 that are capable of engaging the users 112 naturally and/or interactively. The avatar generation service 202 may be communicatively coupled to the content delivery platform 102 and/or the AI engine 120. The avatar generation service 202 may generate the AI avatar 204 as a function of the curated topic set 128 and/or the curated question and answer set 130, which may enable the AI avatar 204 to deliver context-specific responses and/or present topic-aligned interactions. The avatar generation service 202 may be activated in response to configuration parameters of the content delivery platform 102 and/or may be dynamically selected based on user profile attributes. The avatar generation service 202 may update, re-render, and/or refine the AI avatar 204 as additional curated topics are extracted, approved, and/or integrated into the content delivery platform 102, according to one embodiment.

The AI avatar 204 may be a digitally rendered, intelligent interface entity generated by the avatar generation service 202. The AI avatar 204 may be configured to receive and/or respond to the user questions 124 using curated answers selected by the AI engine 120. The AI avatar 204 may serve as the front-facing interactive medium through which the user 112 receives responsive communications, including but not limited to text, audio, slide presentations 210, and/or downloadable documents 212. The AI avatar 204 may present follow-up questions and/or guide the users 112 through topic-specific information pathways. The AI avatar 204 may operate using, including but not limited to verbal, textual, and/or visual modes, depending on the interface in use (e.g., web portal 106 or mobile app 108), according to one embodiment.

The voice/text input 206 may be a multimodal data capture mechanism configured to enable the user 112 to submit queries through spoken and/or typed natural language. The voice/text input 206 may include a speech-to-text converter and/or a natural language preprocessing module adapted to convert audio-based and/or free-text inputs into the tokenized linguistic units 138. The voice/text input 206 may transmit the tokenized inputs to the content delivery platform 102, where the AI engine 120 may communicate with the large language model 146 to process the user question 124. The voice/text input 206 may support complex, multipart questions and may facilitate parsing of multiple embedded queries. The parsed queries may be individually compared against the curated question and answer set 130 within the vector embedding space 136 to identify the corresponding best answers 144 based on the minimum distance 140, according to one embodiment.

The user interface 208 may be a graphical, voice-enabled, and/or touch-based interface configured to allow the users 112 to engage with the AI avatar 204 and various components of the content delivery platform 102. The user interface 208 may support, including but not limited to, natural language inputs, selection-based commands, feedback collection, downloadable document access, and/or real-time interactions with the generated AI avatar 204. The user interface 208 may be implemented across multiple environments, including the tablet 104, the desktop 106, and the mobile device 108. The user interface 208 may include interactive input fields, response display panels, and/or visual content presentation regions configured to display, including but not limited to, the slide presentations 210, the downloadable documents 212, and/or curated answers. The user interface 208 may further display suggested follow-up questions derived from the curated question and answer set 130 and may present confirmation prompts related to adverse event detection and/or product quality issue reporting, which may be based on analysis performed by the large language model 146 integrated within the AI engine 120, according to one embodiment.

The slide presentation 210 may be a series of visual frames generated by the content delivery platform 102 and presented to the user 112 by the AI avatar 204 as the responsive communication 142 associated with the selected best answer 144. The slide presentation 210 may include the approved content 114 arranged in a semantically optimized order. The sequence of slides in the slide presentation 210 may be determined based on minimum vector distances between slide topics within the vector embedding space 136, as calculated by the large language model 146. This ordering may enable a personalized and/or logically flowing presentation tailored to the context of the user question 124. The AI avatar 204 may sequentially display each slide through the user interface 208 and may prompt the user for interactive input, expansion options, and/or follow-up engagement, according to one embodiment.

The downloadable document 212 may be a static and/or dynamic file containing the approved content 114 presented in response to the user question 124. The downloadable document 212 may be generated by and/or selected from the curated content set as a function of its proximity to the selected best answer 144 in the vector embedding space 136. The downloadable document 212 may include text, images, graphs, references, and/or tables, and may be formatted as a PDF, Word document, and/or HTML page. The user 112 may be presented with an option to download the document for offline reference, which may enable retention and/or reuse of important content, according to one embodiment.

The peer-to-peer (P2P) sharing module 214 may be a component of the content delivery platform 102 configured to enable the user 112 to securely share the approved content 114 with peers. Accessible via the user interface 208 and activated through the dropdown menu 216, the P2P sharing module 214 may retrieve the responsive communication 142, including but not limited to, the best answer 144, the slide presentation 210, and/or downloadable document 212, which may be generated by the AI avatar 204. The peer-to-peer (P2P) sharing module 214 may then generate the QR code 218 and/or the tokenized invite link 220 to allow access to the shared content, according to one embodiment.

The dropdown menu 216 may be a user interface element that may include, but not be limited to, content sharing, document download, follow-up question prompts, and/or feedback options. The dropdown menu 216 may allow the user 112 to select content items identified as relevant to the user question 124, and may interface with the P2P sharing module 214 to enable sharing of the QR code 218 and/or the tokenized invite link 220. The dropdown menu 216 may be dynamically populated based on the curated question and answer set 130 and content relevance calculated by the LLM 146, according to one embodiment.

The QR code 218 may be a scannable graphical object generated by the content delivery platform 102 to enable access to a specific piece of the approved content 114. The QR code 218 may be created in response to the user's 112 sharing request and may encode a reference to the content item selected from the curated question and answer set 130. The QR code 218 may be distributed to a peer who, upon scanning, is directed to view the same content within the content delivery platform 102, according to one embodiment.

The tokenized invite link 220 may be a URL and/or hyperlink generated by the content delivery platform 102 that may include a unique token referencing the approved content 114. The tokenized invite link 220 may be used to provide secure access to the approved content 114 and may be distributed via external digital communication platforms, including but not limited to email, SMS, and/or messaging applications. The tokenized invite link 220 may allow a peer user to access the content delivery platform 102 and view the approved content 114 associated with the user question 124. The tokenized invite link 220 may be generated in conjunction with the QR code 218, and may be selectable via the dropdown menu 216 presented within the user interface 208. The tokenized invite link 220 may be used in a two-click P2P sharing method managed by the P2P sharing module 214, enabling efficient and minimal-step transmission of relevant information between the users 112 of the content delivery platform 102, according to one embodiment.

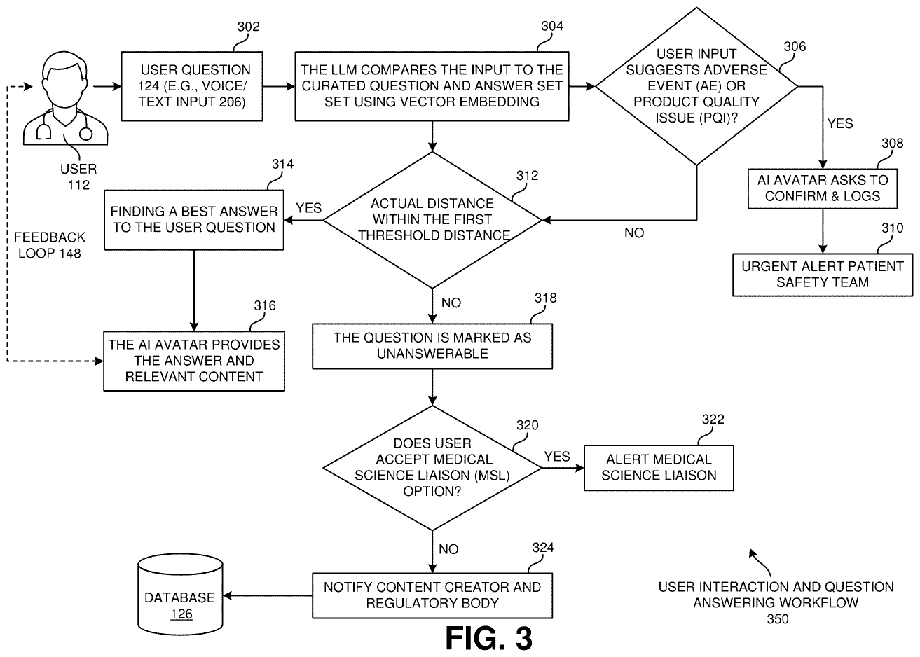

FIG. 3 is a user interaction and question answering workflow 350 of the AI-powered concierge system of FIG. 1, according to one embodiment.

The process may begin when the user 112 provides the user question 124, including but not limited to, the voice/text input 206. The AI engine 120 may identify this input as the user question 124. The AI engine 120 may receive the user question 124 through the content delivery platform 102 and convert the input into the tokenized linguistic units 138, which may then be processed using the large language model 146, according to one embodiment.

The large language model 146 may compare the semantic representation of the user question 124 to entries in the curated question and answer set 130, which may be derived from the curated topic set 128. The comparison may occur in the vector embedding space 136. If the actual distance 402 between the user question 124 and any entry in the curated question and answer set 130 is within the first threshold distance 134, the AI engine 120 may determine that the question is answerable, according to one embodiment.

In response to determining that the question is answerable, the AI engine 120 may retrieve the best answer 144 from the curated question and answer set 130. The best answer may be the entry in the vector embedding space 136 with the minimum distance to the input question. A responsive communication 142 may then be delivered to the user 112 via the AI avatar 204, which may present the answer in text, voice, slide, and/or document format, depending on what is most relevant to the user question 124, according to one embodiment.

If the user question 124 is determined to be unanswerable (i.e., the actual distance 402 exceeds the threshold distance 134), the question may be tagged as unanswerable and marked in the database 126. An alert may then be transmitted to the content creator 116 and the regulatory body 118 for further review and/or approval of the new content 110, according to one embodiment.

If the AI engine 120 detects trigger terms suggestive of an adverse event (AE) and/or product quality issue (PQI), the AI avatar 204 may initiate a confirmation dialog with the user 112 via the content delivery platform 102. Upon receiving confirmation from the user 112, the AI engine 120 may log the event in the database 126 and transmit the alert to a designated patient safety team. Additionally, the AI avatar 204 may present the user 112 with the option to be contacted by a medical science liaison (MSL) for further assistance, according to one embodiment.

The avatar 204 may further present a follow-up question to determine if the user 112 would like additional information about a closely related subtopic. This follow-up may also be generated using the large language model 146 based on the proximity of related entries in the vector embedding space 136.

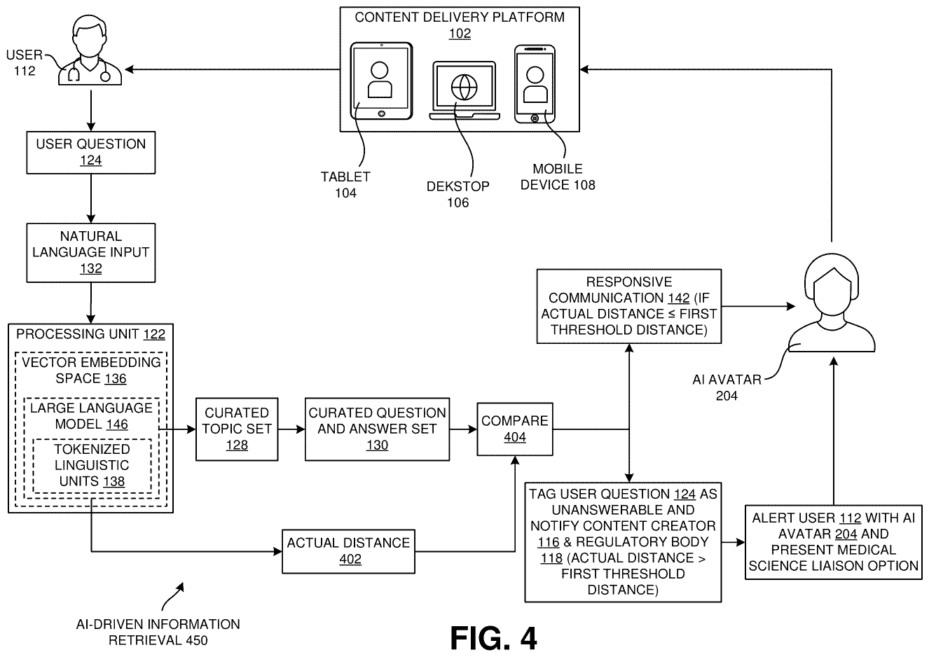

FIG. 4 is an AI-driven information retrieval architecture 450 of the content delivery platform 102 of FIG. 1, according to one embodiment. FIG. 4 illustrates the content delivery platform 102, the tablet 104, the desktop 106, the mobile device 108, the user 112, the content creator 116, the regulatory body 118, the AI engine 120, the processing unit 124, the user question 124, the curated topic set 128, the curated question and answer set 130, the natural language input 138, the vector embedding space 136, the tokenized linguistic units 138, the the responsive communication 142, the large language model 146, the AI avatar 204, an actual distance 402, and a compare 404, according to one embodiment.

The actual distance 402 may be a computed numerical metric representing the semantic proximity between the user question 124 and each question in the curated question and answer set 130 within the vector embedding space 136. This distance may be calculated using machine learning algorithms embedded in the large language model 146 operating on the tokenized linguistic units 138. The actual distance 402 may be compared to the threshold distance 134 to determine whether the user question 124 is answerable. If the actual distance 402 is less than or equal to the threshold distance 134, the AI engine 120 may identify the best answer 144 from the curated question and answer set 130; otherwise, the question may be marked as unanswerable and flagged for review by the content creator 116 and the regulatory body 118, according to one embodiment.

The content delivery platform 102 may interact with the AI engine 120 and the large language model 146 to generate answers in response to the user question 124. The content delivery platform 102 may begin processing with the user question 124 that may be received via the natural language input 138. The user question 124 may be processed by the AI engine 120, and the tokenized linguistic units 138 may be extracted from the user question 124, according to one embodiment.

The AI engine 120 may perform operations within the vector embedding space 136. The AI engine 120 may use the large language model 146 to convert the tokenized linguistic units 138 into numerical vector values. The numerical vector values may then be compared to vector values associated with questions in the curated question and answer set 130. The curated question and answer set 130 may be organized under the curated topic set 128, according to one embodiment.

The AI engine 120 may calculate the actual distance 402 between the user question 124 and each question in the curated question and answer set 130. The actual distance 402 may then be compared to the first threshold distance. If the actual distance 402 may be determined to be within the first threshold distance, the AI engine 120 may identify the best answer 144 from the curated question and answer set 130 and may generate the responsive communication 142. The responsive communication 142 may be delivered via the content delivery platform 102 and may include approved content 114 that may correspond to the selected best answer 144, according to one embodiment.

If the actual distance 402 may exceed the first threshold distance, the AI engine 120 may tag the user question 124 as unanswerable. A notification may be sent to the content creator 116 and/or the regulatory body 118, which may prompt further content development and/or review. The AI engine 120 may further alert the user 112 with the AI avatar 204 and may present the user 112 with the option to consult a medical science liaison, according to one embodiment.

The content delivery platform 102 may include a desktop 106, a mobile device 108, and a tablet 104, each of which may be configured to facilitate access to the content retrieval functionality. The AI avatar 204 may be configured to present the responsive communication 142 and may interact with the user 112 by delivering the selected best answer 144 in a conversational format, according to one embodiment.

The information retrieval system 450 may use the feedback loop 148 to support dynamic feedback. The feedback loop 148 may help update the curated topic set 128 and the curated question and answer set 130 based on user engagement, content results, and input from the regulatory body 118. The AI engine 120 may improve the curated question and answer set 130 over time by using the feedback from the users 112 and the performance of the answers, which may help increase accuracy and/or relevance, according to one embodiment.

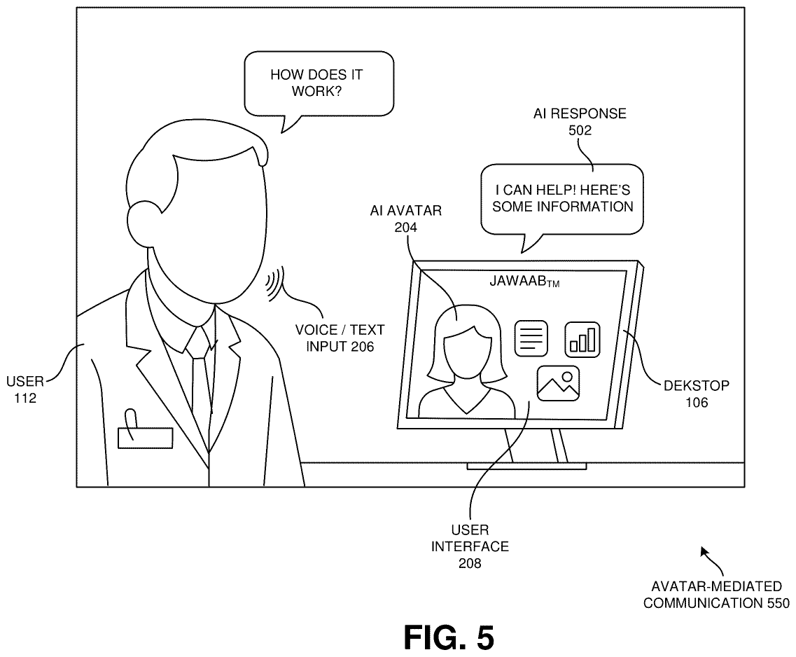

FIG. 5 is an avatar-mediated communication view 550 of the AI-powered concierge system of FIG. 1, according to one embodiment. FIG. 5 illustrates the user 112, the desktop 106, the AI avatar 204, the voice/text input 206, and an AI response 502.

The AI avatar 204 may interact with the user 112 through the desktop 106, which may serve as one of the primary access points to the content delivery platform 102. The user 112 may provide the voice/text input 206 into the AI engine 120 via the desktop 106, initiating the user question 124 directed to the AI avatar 204. The voice/text input 206 may be converted into a standardized text format suitable for the natural language processing 152. The AI avatar 204 may transmit the standardized input to the AI engine 120 (not shown in this figure), which may include the large language model 146 capable of performing semantic analysis in the vector embedding space 136, according to one embodiment.

The AI engine 120 may analyze the user input by generating a vector representation and comparing it to questions from the curated question and answer set 130, which may be stored in the database 126. The semantic proximity between the user's 112 input and the pre-approved questions may be measured using the actual distance 402, and may be evaluated against the predefined threshold distance 134. If the input is determined to be answerable, the AI engine 120 may identify the most relevant response, which may be referred to as the best answer 144, from the curated question and answer sets 130. The selected best answer 144 may then be returned to the AI avatar 204, according to one embodiment.

The AI avatar 204 may render the best answer 144 as the AI response 502, which may be communicated back to the user 112 through the desktop 106 in an interactive, conversational format. The AI response 502 may include a direct answer as well as optional links to the approved content 114, including but not limited to documents, videos, and/or slide presentations 210, depending on the AI engine 120 configuration, according to one embodiment.

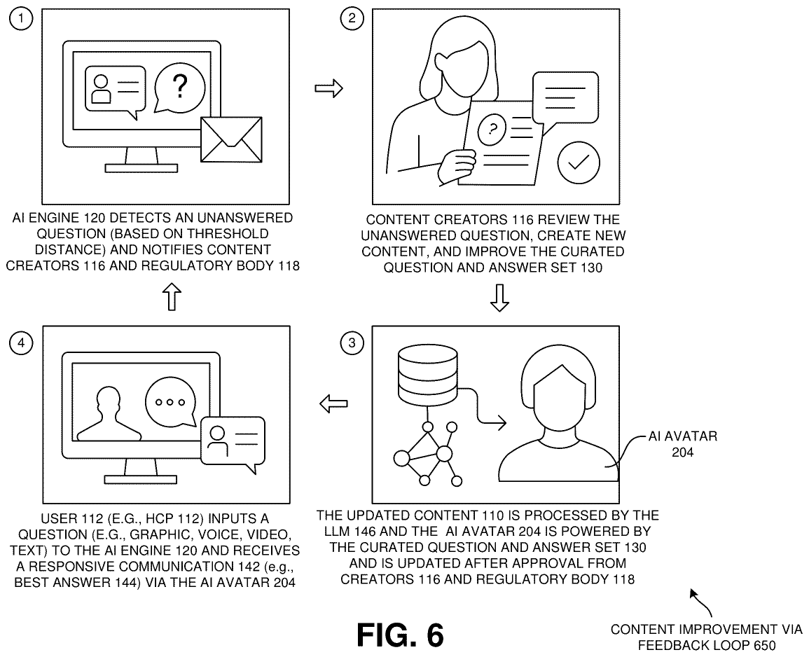

FIG. 6 is a content improvement workflow view 650 of the AI-powered concierge system of FIG. 1, according to one embodiment. FIG. 6 illustrates the content delivery platform 102, the user 112, the approved content 114, the content creator 116, the regulatory body 118, the AI engine 120, the user question 124, the curated question and answer set 130, the responsive communication 142 (e.g., best answer 144), the AI avatar 204, and the feedback loop 148, according to one embodiment.

The process may begin when the user question 124 may be received by the content delivery platform 102 through one of its user interfaces 208, which may include a desktop 106, a mobile device 108, and/or a tablet 104. The user question 124 may be processed by the AI engine 120, which may use the natural language processing 152 and the large language model 146 to convert the question into the tokenized linguistic units 138 that may be embedded within the vector embedding space 136, according to one embodiment.

Within the vector embedding space 136, the AI engine 120 may compare the user question 124 against all entries in the curated question and answer set 130 using the actual distance 402. If the actual distance 402 between the user question 124 and the closest existing question in the curated question and answer set 130 may exceed the threshold distance 134, the AI engine 120 may determine that no suitable match exists, according to one embodiment.

At that point, the user question 124 may be tagged as unanswerable and may be stored in the database 126. The AI engine 120 may then initiate the feedback loop 148 by automatically generating a notification to the content creator 116 and/or the regulatory body 118. This notification may contain the text of the unanswerable question and may also include any contextual data provided by the user 112, including but not limited to prior queries and/or selected categories, according to one embodiment.

Upon receiving the notification, the content creator 116 may review the unanswerable question and, if necessary, may collaborate with the regulatory body 118 to generate new content and/or update existing content. The updated content may be incorporated as new entries into the curated topic set 128 and, subsequently, into the curated question and answer set 130, according to one embodiment.

The newly added question-answer pair may then be processed by the large language model 146 and may be made available to the AI engine 120 for future semantic comparisons. As a result, the next time the same and/or a similar user question 124 may be submitted, it may fall within the defined threshold distance 134 and may trigger the selection of the best answer 144, which may be delivered as the responsive communication 142, according to one embodiment.

The selected best answer 144 may then be presented to the user 112 through the AI avatar 204, which may provide interactive delivery via the content delivery platform 102. This improvement cycle may ensure that the system becomes more robust and/or comprehensive over time by closing gaps in the knowledge base through continuous learning and/or regulated content updates, according to one embodiment.

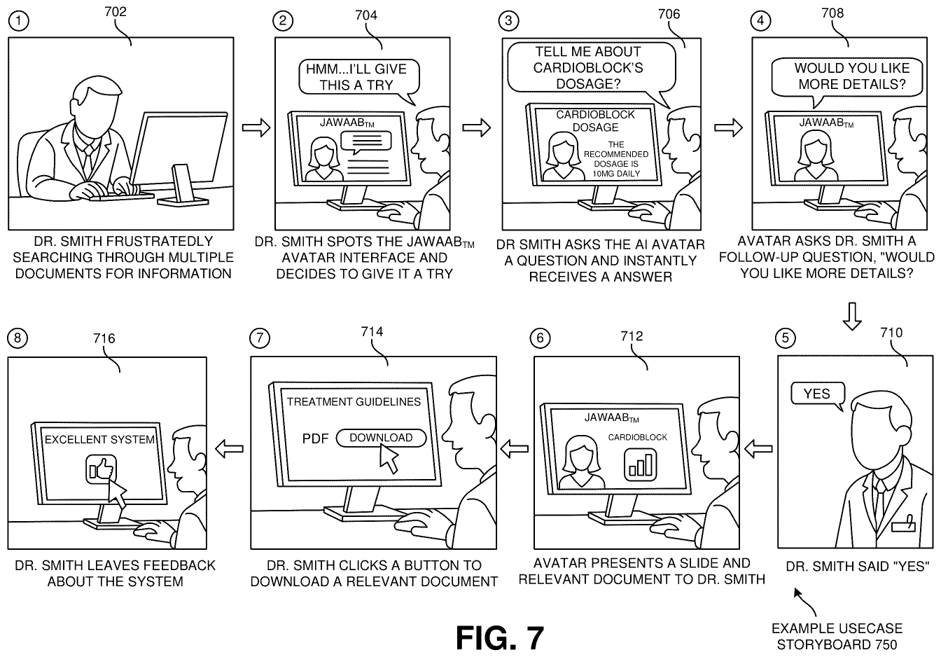

FIG. 7 illustrates a narrative-driven example 700 showing how a user, named Dr. Smith, experiences the benefits of the AI-powered content delivery platform of FIG. 1, according to one embodiment.

In 702, before the implementation of the AI-powered content delivery platform 102, Dr. Smith may be feeling frustrated because he may be trying to find accurate and reliable information about a pharmaceutical product by searching through multiple static documents, references, and clinical files. The traditional process may be inefficient, requiring manual searches across fragmented resources that may not provide clear and/or immediate answers. Dr. Smith may waste valuable time trying to locate specific dosage guidelines and/or treatment protocols, which may result in delay, confusion, and/or dissatisfaction, according to at least one embodiment.

In 704, Dr. Smith may encounter the AI avatar interface, branded as Jawaab™, which is a feature of the content delivery platform. Upon discovering the interface, Dr. Smith may decide to give the system a try by asking a medical question, “Tell me about CardioBlock's dosage?” The AI avatar may receive the user question and begin processing it using the AI engine and the large language model. This marks a transition from inefficient manual search to AI-assisted query resolution, according to at least one embodiment.

In 706, the AI avatar may respond to Dr. Smith's question in real time with a concise and accurate best answer. The avatar may state the recommended dosage for CardioBlock as “10 mg daily,” based on the curated question and answer set derived from approved content. This interaction may demonstrate the responsiveness and clarity of the system and the ability of the AI engine to provide relevant answers efficiently, according to at least one embodiment.

In 708, the AI avatar may continue the conversation by asking Dr. Smith whether more detailed information is desired, such as related treatment guidelines or supportive materials. This step highlights the AI avatar's conversational capabilities and adaptive interaction design. By proactively offering follow-up information, the system may deepen engagement and ensure that users receive comprehensive support tailored to their needs, according to at least one embodiment.

In 710, Dr. Smith may respond “YES” to the AI avatar's prompt and may be presented with a slide presentation comprising relevant clinical guidance. The slides may be selected based on proximity to the best answer in the vector embedding space, ensuring contextual alignment with the original query, according to at least one embodiment.

In 712, Dr. Smith may be given the option to download a document for future reference. This downloadable document may be determined as a function of its minimum distance to the selected best answer in the vector embedding space. The AI avatar may provide a download button, streamlining access to approved content in a user-friendly format, according to at least one embodiment.

In 714, Dr. Smith may click the button and successfully download the relevant document. This download may allow the user to store or share the content for use in patient care or internal decision-making. The system's ability to deliver tangible outputs may reinforce its value to clinicians and healthcare professionals seeking validated resources, according to at least one embodiment.

In 716, Dr. Smith may leave feedback on the interaction with the AI avatar 204, completing the user session. This feedback may be captured by the feedback loop 148 and stored in the database 126 as a tagged interaction, indicating whether the user question was answered, unanswerable, and/or required revision. The feedback may be used to improve future responses by refining the curated question and answer set through the LLM, according to at least one embodiment.

### Additional Embodiments

In one embodiment, the content delivery platform 102 may be integrated into a voice-first interface such as a smart speaker or in-vehicle assistant. For example, a healthcare provider 112 traveling between clinics may submit a user question 124 through a spoken query using a smart speaker interface, functioning as a voice/text input 206. The AI engine 120 receives and processes this query through natural language processing 152, generates tokenized linguistic units 138, and performs semantic comparison operations within the vector embedding space 136. The best answer 144 is selected from the curated question and answer set 130, and the responsive communication 142 is returned audibly via an AI avatar 204.

In another embodiment, the AI avatar 204 may be rendered through augmented reality (AR) devices such as AR glasses or headsets. In this use case, the avatar appears within the field of view of the healthcare provider 112, enabling real-time access to approved content 114 while the provider is engaged in clinical procedures or training. The AI avatar 204 may present this content in a slide presentation 210, where the sequence of slides is arranged as a function of minimum distance 140 between slides in the vector embedding space 136, providing a semantically coherent educational experience.

In yet another embodiment, the content delivery platform 102 supports multimodal input, such as image uploads. A user 112 may upload an image of a pill bottle, a skin reaction, or a handwritten note through the user interface 208 on a mobile device 108 or desktop 106. The image is processed to extract embedded metadata or keywords, which are then converted into tokenized linguistic units 138 and matched to curated content using the large language model 146. The best answer 144, along with a downloadable document 212, may be presented to the user 112 through the AI avatar 204.

In a further embodiment, the system is adapted for use in consumer-facing environments such as retail pharmacies. The content delivery platform 102 may be installed in a kiosk interface at the pharmacy, where a non-professional user 112 can submit a voice or text query through input 206. The system restricts the curated question and answer set 130 to consumer-approved content and provides a responsive communication 142 through the AI avatar 204, with enhanced compliance auditing recorded in the database 126.

In another embodiment, peer-to-peer content sharing is facilitated via the P2P sharing module 214. After receiving a helpful answer 144, the user 112 may activate a sharing option through the dropdown menu 216, prompting the system to generate a QR code 218 and a tokenized invite link 220. These sharing mechanisms allow secure and tracked dissemination of the same responsive communication 142 to another healthcare provider who can then view the approved content 114 via the content delivery platform 102.

In an embodiment oriented toward pharmacovigilance, the large language model 146 analyzes the user question 124 to detect trigger words indicative of an adverse event. If such terms are identified, the avatar 204 presents a dialog prompt through the interface 208 confirming whether the event was experienced. Upon user confirmation, the system stores the response in the database 126 and notifies a designated patient safety team, thereby supporting automated adverse event escalation.

In a further embodiment, the curated question and answer set 130 is continuously refined by a feedback loop 148. Each interaction is tagged with a feedback label stored in the database 126, such as “answered,” “unanswered,” or “requires revision. ” The large language model 146 uses this tagged feedback to iteratively improve the curated topic set 128 and answer set 130, ensuring that future queries have improved match rates within the vector embedding space 136.

In yet another embodiment, the content delivery platform 102 supports user-specific personalization. User interactions are anonymized and stored in the database 126, allowing the large language model 146 to tailor responses based on medical specialty, frequently accessed topics, or preferred formats (e.g., slides 210 vs. documents 212). This personalization ensures that each responsive communication 142 delivered via the AI avatar 204 is contextually optimized for the end user 112.

In one embodiment, the AI engine 120 integrates multiple large language models 146 specialized for different tasks. One model may be fine-tuned for regulatory-approved phrasing, while another is optimized for semantic similarity matching. The processing unit 122 orchestrates collaboration between models, ensuring that selected answers 144 meet both semantic relevance and compliance constraints.

Finally, in a multi-turn dialogue embodiment, the AI avatar 204 maintains context across multiple queries. After answering an initial user question 124, the avatar may ask a follow-up question derived from the curated topic set 128 and present options such as “Would you like more details about contraindications?” Each subsequent interaction is semantically mapped in the vector embedding space 136 to refine relevance and continuity in the responsive communication 142.

Although the present embodiments have been described with reference to specific example embodiments, it will be evident that various modifications and changes may be made to these embodiments without departing from the broader spirit and scope of the various embodiments. For example, the various devices and modules described herein may be enabled and operated using hardware circuitry (e.g., CMOS based logic circuitry), firmware, software or any combination of hardware, firmware, and software (e.g., embodied in a non-transitory machine-readable medium). For example, the various electrical structure and methods may be embodied using transistors, logic gates, and electrical circuits (e.g., application specific integrated (ASIC) circuitry and/or Digital Signal Processor (DSP) circuitry).

In addition, it will be appreciated that the various operations, processes and methods disclosed herein may be embodied in a non-transitory machine-readable medium and/or a machine-accessible medium compatible with a data processing system (e.g., data processing device 100). Accordingly, the specification and drawings are to be regarded in an illustrative rather than a restrictive sense.

A number of embodiments have been described. Nevertheless, it will be understood that various modifications may be made without departing from the spirit and scope of the claimed invention. In addition, the logic flows depicted in the figures do not require the particular order shown, or sequential order, to achieve desirable results. In addition, other steps may be provided, or steps may be eliminated, from the described flows, and other components may be added to, or removed from, the described systems. Accordingly, other embodiments are within the scope of the following claims.

It may be appreciated that the various systems, methods, and apparatus disclosed herein may be embodied in a machine-readable medium and/or a machine accessible medium compatible with a data processing system (e.g., a computer system), and/or may be performed in any order.

The structures and modules in the figures may be shown as distinct and communicating with only a few specific structures and not others. The structures may be merged with each other, may perform overlapping functions, and may communicate with other structures not shown to be connected in the figures. Accordingly, the specification and/or drawings may be regarded in an illustrative rather than a restrictive sense.

1. A method comprising,
configuring a content delivery platform to receive approved content, wherein the approved content has been approved by at least one of a content creator and a regulatory body;
coupling the content delivery platform to a Large Language Model (“LLM”) adapted to perform operations in a vector embedding space on a tokenized linguistic units within the approved content:
wherein the approved content comprises at least one of a question, an answer, an image, a video, an audio, a document, a web page, a link and a markup, using the content delivery platform;
characterizing one or more topics in the approved content using the LLM to extract the topics from the approved content;
creating a curated topic set based on identifying and eliminating mutually exclusive topics from the extracted topics, using the LLM;
creating a curated question and answer set comprising mutually exclusive questions and answers from the curated topic set, wherein the mutually exclusive questions and answers are determined as a function of the curated topic set using the LLM;
configuring an avatar generation service to generate an Artificial Intelligence (“AI”) avatar determined as a function of the curated topic set, using the content delivery platform;
providing a user with interactive access to the generated avatar, using the content delivery platform;
receiving at least one of a voice input and a text input comprising a user question directed to the generated AI avatar, using the content delivery platform; determining if the user question is answerable by an answer selected from the curated question and answer set, based on comparing a threshold distance to a distance between the user question and each question from the curated question and answer set in the vector embedding space, using the LLM;
in response to determining the user question is not answerable, marking the user question as unanswerable in a database and sending an indication to at least one of the content creator and the regulatory body that the user question could not be answered; and in response to determining the user question is answerable: finding a best answer to the user question, wherein the best answer is selected from the curated question and answer set as a function of a distance in the vector embedding space, using the LLM; and
directing the generated avatar to interactively answer the user question with the selected best answer to the user question, using the content delivery platform.

2. The method of claim 1,
wherein the method further comprises in response to determining two or more questions are present in the received the voice and the text input, using the LLM, find the best answer to the two or more questions, and
wherein the best answer to the two or more questions is selected from the curated question and answer set as a function of a distance between answers to the two or more questions in the vector embedding space, using the LLM.

3. The method of claim 1,
wherein after directing the generated avatar to interactively answer the user question with the selected best answer to the user question, the generated avatar presents relevant content to the user that is most relevant in context of the user question, and
wherein the relevant content that is determined as a function of the distance between a speculative content item and the selected best answer to the user question in the vector embedding space, using the LLM.

4. The method of claim 3,
wherein the content that is most relevant further comprises a slide presentation, and
wherein the slide presentation comprises a sequence of slides ordered as a function of the minimum distance from slide to slide in the vector embedding space, using the LLM.

5. The method of claim 4,
wherein the content that is the most relevant further comprises a downloadable document selected as a function of the minimum distance from the selected best answer in the vector embedding space, using the LLM, and
wherein the user is presented with the option to download the content to their device for future reference.

6. The method of claim 1,
wherein the method further comprises presenting a follow-up question to the user, and
wherein the follow-up question is directed to determining if the user would like more information about a subtopic closely related to the selected best answer, determined as a function of the selected best answer and the curated question and answer set, using the LLM.

7. A system comprising:
a content delivery platform comprising a user interface configured to receive a user question, and deliver a best answer,
wherein the content delivery platform comprises at least one of a tablet, a desktop, and a mobile device, each configured to receive and transmit the user question to an artificial intelligence engine;
a large language model integrated within the AI engine communicatively coupled to the content delivery platform, the large language model configured to perform operations in a vector embedding space on a tokenized linguistic unit derived from approved content, the AI engine configured to:
process the user question received via at least one of a voice input and a text input from the content delivery platform,
retrieve relevant information from a database comprising the approved content,
wherein the approved content comprises a curated topic set derived from extracted topics using the large language model, and a curated question and answer set comprising mutually exclusive questions and mutually exclusive answers derived from the curated topic set, and
wherein the approved content is presented in one or more formats selected from: an image, a video, an audio file, a document, a web page, a hyperlink, and a markup,
compare a threshold distance to an actual distance between the user question and each question from the curated question and answer set in the vector embedding space,
identify the best answer based on a minimum distance below the threshold distance,
generate the best answer to the user question if the actual distance is determined to be within the threshold distance, and
transmit the best answer to the content delivery platform;
an avatar generation service communicatively coupled to the AI engine and the content delivery platform, the Avatar generation Service is configured to:
generate and manage an AI avatar for interaction with the user through the content delivery platform, and
direct the AI avatar to interactively answer the user question using the selected best answer from the curated question and answer set,
wherein the best answer is selected using the vector embedding space by the AI engine; and
a feedback loop configured to transmit data from user interactions with the artificial intelligence engine via the content delivery platform,
wherein the transmitted data is processed to continuously refine the curated question and answer set and enhance the response generation capabilities of the large language model,
wherein each user question is tagged with a feedback label indicating whether the user question was at least one of: answered, unanswered, and requires revision,
wherein the tagged question is stored in the database for continuous training of the large language model,
wherein the curated question and answer set is updated based on feedback received from at least one of the users, the content creators, and the regulatory body, and
wherein the feedback is processed by the large language model to refine the curated question and answer set over time.

8. The system of claim 7,
wherein the user question is received from at least one of: a voice input and a text input, and
wherein the artificial intelligence engine is configured to convert the voice input and the text input into a standardized format before processing the user question within a vector embedding space.

9. The system of claim 7,
wherein the AI avatar is further configured to present to the user content that is most relevant in the context of the user question, the relevant content comprising a slide presentation and a downloadable document selected as a function of a minimum distance from the selected best answer in the vector embedding space.

10. The system of claim 7,
wherein the feedback loop is further configured to process the tagged user questions to identify recurring patterns in unanswered questions, and
wherein the large language model adjusts the curated question and answer set to improve coverage and enhance the relevance of future responses.

11. The system of claim 7 further comprising:,
a peer-to-peer (P2P) sharing module integrated into the content delivery platform, configured to:
allow the user to share the approved content by selecting a share option from a dropdown menu,
generate a QR code and a tokenized invite link associated with the approved content, and
enable the user to share either the QR code, the tokenized invite link, and both, to provide a peer with access to the content delivery platform through a two-click interaction.

12. A method comprising:
identifying a natural language input in the form of a user question transmitted by a user;
providing the natural language input of the user question to a large language model wherein the large language model operates within a vector embedding space that processes a tokenized linguistic unit derived from medical and pharmacological phraseology;
analyzing the natural language text of the user question using the large language model wherein said large language model compares a first threshold distance to an actual distance between the user question and each question from a curated question and answer set in the vector embedding space;
determining whether the actual distance is within the first threshold distance;
automatically generating a best answer as a responsive communication to the user as an output to the large language model if the actual distance is determined to be within the first threshold distance; and
transmitting the responsive communication to the user.

13. The method of claim 12 further comprising:
analyzing a curated topic set using the large language model to extract relevant topics and relationships; and
creating the curated question and answer set as the output of the large language model's analysis of the curated topic set, wherein the question and answer set is generated based on the identified topics and relationships.

14. The method of claim 12 further comprising:
tagging the user question as an unanswerable question if the actual distance is outside of the first threshold distance;
marking the user question the unanswerable in a database; and
notifying a content creator that the user question is the unanswerable question who will then involve the regulatory body if new content needs approval to answer the previously unanswerable question.

15. The method of claim 12 further comprising:
transmitting the responsive communication to the user in the form of an AI avatar that interacts with the user on a content delivery platform, wherein the AI avatar is generated based on the curated question and answer set and is configured to present the selected best answer to the user interactively through the content delivery platform.

16. The method of claim 12 further comprising:
receiving the user question via at least one of: a voice input, a text input, and a selection from one of the displayed questions on the user interface of the content delivery platform, wherein the large language model is adapted to process each format;
converting the voice input into the text input and analyzing the text input to determine the best answer based on comparing the actual distance and the threshold distance in the vector embedding space;
selecting the best answer to the user question from the curated question and answer set based on both semantic relevance and contextual appropriateness, wherein the semantic relevance is determined by comparing the user question's vector representation with the vectors of questions in the curated question and answer set in the vector embedding space, and the contextual appropriateness is determined by considering the context of the user's specific data; and
updating the curated question and answer set based on a feedback from at least one of the users and the content creators, wherein the feedback is processed by the large language model to refine and improve the curated question and answer set over time, thereby increasing the accuracy and relevance of future responses.

17. The method of claim 12 further comprising:
tagging each user question with the feedback label indicating whether the question was at least one of answered, unanswered, and requires revision;
storing the tagged user question in the database for continuous training of the large language model, wherein the tagged user question is used by the large language model to track the performance of the AI engine and refine its question-answering capabilities;
processing the tagged user question to identify recurring patterns in unanswered questions, wherein the LLM adjusts the curated question and answer set to address identified gaps, improve coverage, and enhance the relevance of future responses; and
detecting and processing multiple user questions within a single input, wherein the large language model analyzes the natural language input to segment the input into individual questions and generates a responsive communication to the user that answers each segmented question using the curated question and answer set, based on the distances between the user questions and the answers in the vector embedding space.

18. The method of claim 12 further comprising:
detecting trigger words indicative of an adverse event in the user's input using the large language model;
generating a dialog box through the AI avatar to confirm whether the user at least one of experienced the event and reported the adverse event;
receiving input from the user in the form of a “YES” or “NO” response;
storing the user's response in the database; and
transmitting an alert to a patient safety team to initiate an action.

19. The method of claim 12 further comprising:
identifying trigger phrases in the user's input suggestive of a product quality issue related to a pharmaceutical product using the large language model;
presenting the dialogue box to the user via the AI avatar to verify if the product quality issue was experienced;
collecting the user's response and storing it in the database; and
informing the patient safety team to take appropriate further action.

20. The method of claim 12 further comprising:
alerting the user that the user question is the unanswerable question if the actual distance is outside of the first threshold distance;
alerting the user that the user question is unanswerable with the AI avatar; and
presenting the user with a medical science liaison option if user question is unanswerable using the AI avatar,
wherein the medical science liaison option prompts the AI engine to alert the medical science liaison that the user has at least one unanswerable question and would like to be contacted by the medical science liaison if the user accepts the medical science liaison option.
